(19) United States
(12) Patent Application Publication
(10) Pub. No.: US 2007/0202070 A1
(43) Pub. Date: Aug. 30, 2007

(54) Moisture Retention Polymer Compound

(76) Inventors: Motoaki Kamachi, Kanagawa (JP); Toyoji Kakuchi, Hokkaido (JP); Toshifumi Satoh, Hokkaido (JP)

Correspondence Address: SUGHRUE MION, PLLC, 2100 PENNSYLVANIA AVENUE, N.W., SUITE 800, WASHINGTON, DC, 20037, US

(21) Appl. No.: 10/594,925

(22) PCT Filed: Mar. 25, 2005
(86) PCT No.: PCT/JP05/06406

§ 371 Date: Sep. 29, 2006

Related U.S. Application Data
(60) Provisional application No. 60/560,605, filed on Apr. 9, 2004.

(30) Foreign Application Priority Data
Mar. 31, 2004 (JP) 2004-105931

(51) Int. Cl. A61K 8/73; C08G 63/91; C08F 20/00

(52) U.S. Cl. 424/70.13; 525/54.2; 525/440

(57) ABSTRACT

The present invention relates to (1) a moisture retention polymer compound comprising a structural unit including a multi-branched polysaccharide derivative in which the hydroxyl group (OH) may be substituted by OR wherein R represents a hydrogen atom, a hydrocarbon having 1 to 30 carbon atoms, or a hydrocarbon having 1 to 30 carbon atoms and a hetero atom, and a structural unit without a multi-branched polysaccharide derivative, (2) an external preparation for skin comprising 0.1 to 99.9% by mass of the polymer compound, which can provide moist feeling to the skin and exhibit an appropriate viscosity and solubility, and (3) a cosmetic product comprising the external preparation for skin.

### CROSS-REFERENCE TO RELATED APPLICATIONS

This is an application filed pursuant to 35 U.S.C. Section 111(a) with claiming the benefit of U.S. provisional application Ser. No. 60/560,605 filed Apr. 9, 2004 under the provision of 35 U.S.C. 111(b), pursuant to 35 U.S.C. Section 119(e)(1).

### TECHNICAL FIELD

The present invention relates to a novel moisture retention polymer compound and, more particularly, to a moisture retention polymer compound which has an effect of preserving the moisture in the skin and is suitable for being used in an external preparation for skin.

### BACKGROUND ART

As a compound having an effect of retaining the moisture of the skin, a moisturizer is often used in an external preparation for skin. Such a moisturizer is blended in an external preparation for skin for the purpose of preventing moisture from evaporating, controlling the moisture in the skin surface, and imparting a moist feeling as well as moisture. The moisture amount in the corneum layer of the skin is closely related to a protective function against a variety of outside stimuli, and plays an important role in anti-aging of the skin, and feelings such as a moist feeling and a smooth feeling. This moisture retention in the corneum layer is generally controlled by NMF (natural moisturizing factor) and sebaceous membrane, however, the function is decreased easily due to aging or outside stimuli. Therefore, it is important for an external preparation for skin, in particular, a cosmetic product, to replenish moisturizing components in the skin. Examples of the moisturizing component for replenishing moisture to the skin include, as components which are the same with or similar to components of the living body, amino acids, peptides, proteins, saccharides, polysaccharides, lipids and glycoproteins.

In particular, a wide variety of compounds are known as saccharides. Examples thereof include alginic acid, pullulan, dextrin, glycogen, mucopolysaccharide, hyaluronic acid, chondroitin sulfate, chitin, chitosan and cellulose. For example, JP-A-1999-180818 (“JP-A” means a publication of an unexamined Japanese paten application) discloses an external preparation for skin containing glycogen derived from a plant, JP-A-2001-89381 discloses an external preparation for skin containing mucopolysaccharide, JP-A-2002-145753 discloses a cosmetic product containing hyaluronic acid, and JP-A-1997-249520 discloses a cosmetic product containing cellulose or starch. However, these naturally-occurring polysaccharides are composed of multiple types of monosaccharides, and the composition ratios are not constant, nor are the structures, therefore it is difficult to control the quality. In addition, when used in an external preparation for skin, such a component often causes aggregation structure where components are inhomogeneously present, to thereby generate gelation or precipitation. Further, there are drawbacks that when such a preparation is applied to the skin, an unpleasant feeling, an offensive odor or the like may be imparted.

In addition, as moisturizing components, many types of synthetic polymers are known. Examples thereof include sodium polyacrylate, polyethyleneimine, polyethylene glycol, polyvinyl alcohol, polyvinyl pyrrolidone, polyacrylamide, polyacrylic acid, polymethacrylic acid, vinylpyridine, silicones and polyoxyethylene. JP-A-2000-143484 discloses water-containing cosmetic sheet containing polyacrylic acid, and JP-A-1997-124878 discloses a gel composition for cosmetic product containing polymer of acrylic acid. Since the molecular weight of synthetic polymers can be easy to control, polymers with various properties or forms can be produced. On the other hand, however, in the case where such a polymer is used in an external preparation for skin, such an external preparation for skin cannot be said to have a sufficient moisture retention property which is comparable to that of a naturally-occurring polysaccharide and a high affinity to the skin to impart sufficient moisture to the skin.

### DISCLOSURE OF THE INVENTION

An object of the present invention is to provide an external preparation for skin which can impart moisture to the skin and can exhibit an appropriate viscosity and solubility. Generally, in an external preparation for skin, a wide variety of compounds such as compounds for imparting a moisture retention property and compounds for imparting viscosity are blended. However, due to difference in solubility between the compounds, separation, precipitation, crystallization or the like occurs, whereby properties of the external preparation for skin or feeling upon using the external preparation for skin is significantly decreased in some cases. Another object of the present invention is to provide an external preparation for skin comprising a polymer that can reduce or avoid these disadvantages.

The present inventors, as a result of intensive studies in order to solve the foregoing problems, found out that by producing a polymer having in a molecule a repeating structural unit that imparts a moisture retention property and further having a unit that imparts viscosity and/or a unit that imparts solubility and the like in the same molecule and blending such a polymer in an external preparation for skin, the external preparation for skin can provide moisture to the skin and further give a good feeling to the skin while retaining an appropriate viscosity and solubility, thus completed the present invention.

In other words, the present invention relates to the following aspects.

[1] A moisture retention polymer compound comprising a structural unit including a multi-branched polysaccharide derivative in which the hydroxyl group (OH) may be substituted by OR wherein R represents a hydrogen atom, a hydrocarbon having 1 to 30 carbon atoms, or a hydrocarbon having 1 to 30 carbon atoms and a hetero atom, and a structural unit without a multi-branched polysaccharide derivative.

[2] The moisture retention polymer compound according to [1], wherein the main chain of the polymer compound consists of a structural unit including a multi-branched polysaccharide derivative.

[3] The moisture retention polymer compound according to [2], which is a polymer compound obtained by copolymerization of a multi-branched polysaccharide derivative having at least two hydroxyl groups and a dicarboxylic acid and/or a diisocyanate.

[4] The moisture retention polymer compound according to [1], having a structural unit including a multi-branched polysaccharide derivative in the side chain of the polymer compound.

[5] The moisture retention polymer compound according to [4], which is a polymer compound obtained by reacting a polymer compound having a carboxyl group and/or an isocyanate group in the main chain with the hydroxyl group (OH) of the multi-branched polysaccharide derivative.

[6] The moisture retention polymer compound according to [1] to [5], wherein the multi-branched polysaccharide derivative is a polymer including as a monomer an anhydrosaccharide and/or an anhydrosaccharide derivative with at least one of the hydroxyl groups (OH) of the anhydrosaccharide being substituted by OR wherein R represents a hydrogen atom, a hydrocarbon having 1 to 30 carbon atoms, or a hydrocarbon having 1 to 30 carbon atoms and a hetero atom.

[7] The moisture retention polymer compound according to [6], wherein the anhydrosaccharide or the anhydrosaccharide portion of the anhydrosaccharide derivative is at least one kind selected from the group consisting of 1,6-anhydrosaccharide, 1,4-anhydrosaccharide, 1,3-anhydrosaccharide, 1,2-anhydrosaccharide, and 5,6-anhydrosaccharide.

[8] The moisture retention polymer compound according to [1] to [7], in which the branching degree of the multi-branched polysaccharide derivative is 0.05 to 1.00.

[9] The moisture retention polymer compound according to [1] to [8], wherein solubility of the polymer compound in water, ethanol or a mixed solution of water and ethanol is 1.0% by mass or higher.

[10] A moisturizer comprising a polymer compound according to any one of [1] to [9].

[11] An external preparation for skin comprising 0.1 to 99.9% by mass of a polymer compound according to any one of [1] to [9].

[12] A cosmetic product containing the moisturizer according to [10].

[13] A cosmetic product containing an external preparation for skin according to [11].

The polymer compound of the present invention is a polymer compound having a structural unit including a multi-branched polysaccharide derivative and a structural unit without a multi-branched polysaccharide derivative. With regard to the multi-branched polysaccharide derivative, the hydroxyl group OH of the multi-branched polysaccharide may be substituted by OR wherein R represents a hydrogen atom, a hydrocarbon having 1 to 30 carbon atoms, or a hydrocarbon having 1 to 30 carbon atoms and a hetero atom.

More specifically, it is a polymer compound having a multi-branched polysaccharide derivative either in the main chain or in the side chain.

As an example of the case where the main chain of the polymer compound is composed of a structural unit including a multi-branched polysaccharide derivative, a polymer obtained by reacting multiple hydroxyl groups in the multi-branched polysaccharide derivative with a dicarboxylic acid compound, a diisocyanate compound or the like can be exemplified.

In the case of a polymer compound having a structural unit including a multi-branched polysaccharide derivative in the side chain, for example, a multi-branched polysaccharide derivative may be bound through an appropriate spacer to the main chain of the polymer compound. Examples of the spacer include an organic group having 1 to 30 carbon atoms which may have a hetero atom. Examples of the hetero atom in such an organic group include oxygen, nitrogen, sulfur, silicon and phosphorous, and examples of the organic group including a hetero atom include organic groups which have a hydrocarbon group, carbonyl group, urethane group, peptide group, sulfonyl group, phosphate group or the like as part or the whole of the spacer.

More specifically, the one obtained by reacting the hydroxyl group of the multi-branched polysaccharide derivative with the carboxyl group of a polymer obtained by copolymerization of acrylic acid, methacrylic acid or the like, or with the isocyanate group of a polymer obtained by copolymerization of isocyanate ethyl methacrylate or the like can be exemplified.

Examples of the synthetic method of the polymer compound of the present invention include a method of performing condensation polymerization by mixing a multi-branched polysaccharide derivative with a compound having in the molecule two functional groups that react with a hydroxyl group. For example, by mixing a compound selected from dicarboxylic acids or diisocyanates (e.g., diisocyanate hexane), with a multi-branched polysaccharide at an appropriate ratio to react with each other, polyurethane polymer compound including a multi-branched polysaccharide in the main chain can be obtained.

In addition, a method of synthesizing a monomer including a polymerizable functional group and a multi-branched polysaccharide derivative, and performing condensation polymerization, radical polymerization, ionic polymerization, ring-opening polymerization, block polymerization or graft polymerization of this monomer solely or together with another monomer, etc. can be exemplified.

Further, a method of preparing a polymer having a functional group in advance and thereafter reacting a multi-branched polysaccharide derivative with the foregoing functional group to thereby bind with each other, for example, a method for obtaining a polymer compound having a multi-branched polysaccharide derivative in the side chain by mixing methyl methacrylate with isocyanate ethyl methacrylate at an appropriate ratio, obtaining a polymethacrylic acid copolymer by radical polymerization and reacting the hydroxyl group of a multi-branched polysaccharide derivative with the isocyanate group of the polymethacrylic acid copolymer, can be exemplified. However, the method is not limited to these methods.

The multi-branched polysaccharide constituting the skeleton of the multi-branched polysaccharide derivative of the present invention is a compound composed of one kind or multiple kinds of saccharides as the structural components. The structure is a dendritic structure having many branching points wherein three or more sites binding with adjacent saccharide molecules exist in one saccharide molecule having multiple hydroxyl groups. Generally, a polysaccharide that exists in a variety of types as a natural substance is a linear polysaccharide or a branched polysaccharide having not so many branching points, and both types have its main chain. A multi-branched polysaccharide is a compound wherein its main chain cannot be clearly distinguished due to presence of many branching points.

Examples of the saccharide constituting the multi-branched polysaccharide of the present invention may include, as a pentose, ribose and deoxyribose, and as a hexose, glucose, fructose and galactose.

As a production method of the multi-branched polysaccharide of the present invention, a conventionally known method can be applied. For example, as a production method by a chemical synthesis method, a production method of obtaining a multi-branched polysaccharide by polymerizing a derivative of a monosaccharide as a monomer can be exemplified. Examples thereof include, as disclosed in JP-A-2003-252904, a production method of obtaining a multi-branched polysaccharide by ionic polymerization using an anhydrosaccharide as a monomer.

In this case, examples of the anhydrosaccharide may include, for example, 1,6-anhydrosaccharide, 1,4-anhydrosaccharide, 1,3-anhydrosaccharide, 1,2-anhydrosaccharide, 5,6-anhydrosaccharide, etc., and a derivative thereof. Specific examples may include 1,6-anhydro-β-D-glucopyranose, 1,6-anhydro-β-D-mannopyranose, 1,6-anhydro-β-D-galactopyranose, 1,6-anhydro-β-D-allopyranose, 1,6-anhydro-β-D-altropyranose, 1,4-anhydro-β-D-ribopyranose, 1,4-anhydro-α-D-xylopyranose, 1,4-anhydro-α-L-arabinopyranose, 1,4-anhydro-α-D-lyxopyranose, 1,3-anhydro-β-D-glucopyranose, 1,3-anhydro-β-D-mannopyranose, 1,2-anhydro-α-D-glucopyranose, 1,2-anhydro-β-D-mannopyranose, 5,6-anhydro-α-D-glucopyranose, etc., and a derivative thereof.

In addition, as the ionic polymerization, cation polymerization and anion polymerization can be exemplified. Further, there is a production method of obtaining a multi-branched polysaccharide by using an animal, plant or microorganism, or by using a method of raising, cultivating or culturing it. In this case, a multi-branched polysaccharide with a desired branching degree can be directly used. However, as a method of producing a multi-branched polysaccharide whose branching degree has been increased, for example, by a production method as disclosed in JP-A-1996-41104, the branching degree can be increased so that a branched polysaccharide may become a multi-branched polysaccharide.

The multi-branched polysaccharide derivative of the present invention has a structure in which part and/or all of the hydroxyl groups in the multi-branched polysaccharide that constitutes the skeleton have been substituted by substituent (s). Examples include a compound in which at least one of the hydroxyl groups OH of the multi-branched polysaccharide has been substituted by OR, wherein R represents a hydrogen atom, a hydrocarbon having 1 to 30 carbon atoms, or a hydrocarbon having 1 to 30 carbon atoms which has a hetero atom.

Examples of the R may include, for example, a methyl group, ethyl group, propyl group, isopropyl group, butyl group, 1-methylpropyl group, 2-methylpropyl group, pentyl group, 1-methylbutyl group, 2-methylbutyl group, 3-methylbutyl group, 1-ethylpropyl group, hexyl group, 1-methylpentyl group, 2-methylpentyl group, 3-methylpentyl group, 4-methylpentyl group, 1-ethylbutyl group, 2-ethylbutyl group, heptyl group, 1-methylhexyl group, 2-methylhexyl group, 3-methylhexyl group, 4-methylhexyl group, 5-methylhexyl group, 1-ethylpentyl group, 2-ethylpentyl group, 3-ethylpentyl group, octyl group, 1-methylheptyl group, 2-methylheptyl group, 3-methylheptyl group, 4-methylheptyl group, 5-methylheptyl group, 6-methylheptyl group, 1-ethylhexyl group, 2-ethylhexyl group, 3-ethylhexyl group, 4-ethylhexyl group, 1-propylpentyl group, 2-propylpentyl group, nonyl group, decyl group, undecyl group, 10-undecenyl group, dodecyl group, tridecyl group, tetradecyl group, pentadecyl group, hexadecyl group, 9-hexadecenyl group, heptadecyl group, octadecyl group, isostearyl group, cis-9-octadecenyl group, 11-octadecenyl group, cis-cis-9,12-octadecadienyl group, 9,12,15-octadecatrienyl group, 6,9,12-octadecatrienyl group, 9,11,13-octadecatrienyl group, nonadecyl group, 2,6,10,14-tetramethyl pentadecyl group, icosanyl group, 8,11-icosadienyl group, 5,8,11-icosatrienyl group, 5,8,11,14-icosatetraenyl group, 3,7,11,15-tetramethylhexadecyl group, heneicosanyl group, docosanyl group, acetyl group, propionyl group, isopropionyl group, butyryl group, 1-methylpropionyl group, 2-methylpropionyl group, valeryl group, 1-methylbutyryl group, 2-methylbutyryl group, 3-methylbutyryl group, 1-ethylpropionyl group, hexanoyl group, 2-methylpentanoyl group, 3-methylpentanoyl group, 4-methylpentanoyl group, 2-ethylbutanoyl group, heptanoyl group, 2-methylhexanoyl group, 3-methylhexanoyl group, 4-methylhexanoyl group, 2-ethylpentanoyl group, 3-ethylpentanoyl group, octanoyl group, 2-methylheptanoyl group, 3-methylheptanoyl group, 4-methylheptanoyl group, 5-methylheptanoyl group, 6-methylheptanoyl group, 2-ethylhexanoyl group, 3-ethylhexanoyl group, 4-ethylhexanoyl group, 2-propylpentanoyl group, nonanoyl group, decanoyl group, undecanoyl group, 10-undecenoyl group, dodecanoyl group, tridecanoyl group, tetradecanoyl group, pentadecanoyl group, hexadecanoyl group, 9-hexadecenoyl group, heptadecanoyl group, octadecanoyl group, isostearyl group, cis-9-octadecenoyl group, 11-octadecenoyl group, cis, cis-9,12-octadecadienoyl group, 9,12,15-octadecatrienoyl group, 6,9,12-octadecatrienoyl group, 9,11,13-octadecatrienoyl group, nonadecanoyl group, 2,6,10,14-tetramethylpentadecanoyl group, icosanoyl group, 8,11-icosadienoyl group, 5,8,11-icosatrienoyl group, 5,8,11,14-icosatetraenoyl group, 3,7,11,15-tetramethylhexadecanoyl group, heneicosanoyl group, docosanoyl group, phosphatidyl group and the like. Further, a compound in which R has been bound with an isocyanate group and the like are also included.

As the synthetic method of the multi-branched polysaccharide derivative of the present invention, for example, a method of synthesizing a multi-branched polysaccharide derivative by modifying a hydroxyl group of a saccharide with a desired functional group after obtaining a multi-branched polysaccharide for constituting the skeleton, or a method of synthesizing a multi-branched polysaccharide derivative by polymerization after modifying a hydroxyl group of an anhydrosaccharide with a desired functional group to serve as a monomer of a multi-branched polysaccharide may be employed.

With regard to the modification of the hydroxyl group, for example, a method by a common esterification reaction using a carboxylic acid derivative can be employed. Specific examples include a method of performing reaction by using an acid chloride or a mixed acids anhydride prepared from corresponding carboxylic acid(s), a method of performing reaction by using carbodiimide, a method of using an acid anhydride and the like. Further, in the case of a compound having an isocyanate group, a modification method of forming an urethane bond by heating or by using a catalyst such as a tin-based catalyst or an amine-based catalyst, and the like can be employed.

The branching degree of the multi-branched polysaccharide constituting the skeleton of the multi-branched polysaccharide derivative of the present invention is 0.05 to 1.00, preferably 0.2 to 1.0, more preferably 0.4 to 1.0. The multi-branching degree in this case is generally calculated with Frechet formula as shown below.
Branching Degree=(number of dendritic units+number of polymer ends)/(number of dendritic units+number of polymer ends+number of linear chain units)

Examples of the method of blending the polymer compound to be used in the present invention into an external preparation for skin include a method of producing an external preparation for skin by mixing the polymer compound as a solid, powder or semisolid, a method of producing an external preparation for skin by mixing the polymer compound as an aqueous solution, a method of producing an external preparation for skin by mixing the polymer compound as a solution of an alcohol or an appropriate solvent and a method of adding the polymer compound to an external preparation for skin by a known method.

The polymer compound to be used in the present invention can be isolated, for example, in a form of powder, alternatively, a solution obtained in the process of the production can be directly blended in an external preparation for skin. In addition, for a water-based external preparation for skin, generally, it is preferable that the polymer compound exhibit a high solubility in water. Specifically, it is preferred that the solubility in water, ethanol, or a mixed solution of water and ethanol of the polymer compound be 1.0% by mass or higher.

The polymer compound to be used in the present invention, as long as the blending amount does not hinder formation of the external preparation for skin, can be blended in an amount of 0.01 to 100% by mass, preferably at 0.1 to 99.9% by mass, more preferably at 1% to 50% by mass. If the blending amount is less than 0.01% by mass relative to the external preparation for skin, adhesiveness and affinity to the skin are not sufficient, and a feeling such as a moisturizing feeling to the skin cannot be obtained sufficiently in some cases. It can be blended within such a range that essential functions of the external preparation for skin may not be impaired.

The term “external preparation for skin” used in the present invention includes cosmetics, detergents, bath agents, soaps and the like which are used in direct contact with the skin.

Examples of the external preparation for skin of the present invention include in a wide sense, for example, skin milk, skin cream, foundation cream, massage cream, cleansing cream, shaving cream, cleansing foam, skin lotion, lotion, facial mask, lip rouge, rouge, eye shadow, manicure, soap, body shampoo, hand soap, shampoo, hair conditioner, hair tonic, hair treatment, hair cream, hair spray, hair growth tonic, baldness remedy, hair dye, shmaltz, hair remover, anti-dandruff lotion, toothpaste, artificial teeth adhesive, gargle, permanent wave agent, curling agent, styling agent, ointment, cataplasm, tape, bath agent, a diaphoretic, sun protectant and the like, and any type is included as long as it is used in contact with the skin. In particular, a preferred use of the external preparation for skin of the present invention is a cosmetic product. In addition, the user may be anyone regardless of sex or age. Further, products to be used in contact with the skin of animals other than human are also included.

Examples of the form of the present invention include many forms such as powder, granule, tablet, gel and foam, as well as solid, liquid, semisolid and gas.

In the external preparation for skin of the present invention, as a component other than the polymer compound, components generally used in an external preparation for skin can be blended within the range that does not impair the effect of this invention, as needed. Examples of components include compounds blendable in cosmetic products, compounds serving as raw materials for quasi-drug products, drug products and medicinal additives.

Examples of such an ingredient include hydrocarbons such as ozokerite, α-olefin oligomer, light isoparaffin, light liquid isoparaffin, squalene, squalane, synthetic squalane, phytosqualane, ceresin, paraffin, polyethylene powder, polybutene, microcrystalline wax, liquid isoparaffin, liquid paraffin, mineral oil and vaseline;

natural waxes such as jojoba oil, carnauba wax, candelilla wax, rice bran wax, shellac, lanolin, mink sebaceous wax, spermaceti wax, sugarcane wax, sperm whale oil, beeswax and montan wax, natural fats and fatty oils such as avocado oil, almond oil, olive oil, extra virgin olive oil, sesame seed oil, rice bran oil, rice oil, rice germ oil, corn oil, safflower oil, soybean oil, maize oil, rape seed oil, persic oil, palm kernel oil, palm oil, castor oil, sunflower oil, high oleic sunflower oil, grape seed oil, cottonseed oil, coconut oil, hydrogenated coconut oil, beef tallow, hydrogenated oil, horse oil, mink oil, yolk oil, yolk fat oil, rose hip oil, kukui nut oil, evening primrose oil, wheat germ oil, peanut oil, Camellia jeponica oil, Camellia kissi oil, cacao butter, Japan wax, beef bone tallow, nest's-foot oil, swine tallow, equine tallow, ovine tallow, shea butter, macadamia nut oil and meadow foam seed oil;

fatty acids such as lauric acid, myristic acid, palmitic acid, stearic acid, behenic acid, oleic acid, linoleic acid, linolenic acid, γ-linolenic acid, isostearic acid, 12-hydroxystearic acid, undecylenic acid and coconut oil fatty acid;

higher alcohols such as isostearyl alcohol, octyl dodecanol, hexyl decanol, cholesterol, phytosterol, lauryl alcohol, myristyl alcohol, cetyl alcohol, stearyl alcohol, oleyl alcohol, behenyl alcohol and cetostearyl alcohol;

alkylglyceryl ethers such as batyl alcohol, chimyl alcohol, serachyl alcohol and isostearyl glyceryl ether;

esters such as isopropyl myristate, butyl myristate, isopropyl palmitate, ethyl stearate, butyl stearate, ethyl oleate, ethyl linoleate, isopropyl linoleate, cetyl caprylate, hexyl laurate, isooctyl myristate, decyl myristate, myristyl myristate, cetyl myristate, octadecyl myristate, cetyl palmitate, stearyl stearate, decyl oleate, oleyl oleate, cetyl ricinoleate, isostearyl laurate, isotridecyl myristate, isocetyl myristate, isostearyl myristate, octyldodecyl myristate, 2-ethylhexyl palmitate, isocetyl palmitate, isostearyl palmitate, 2-ethylhexyl stearate, isocetyl stearate, isodecyl oleate, octyldodecyl oleate, octyldodecyl ricinoleate, ethyl isostearate, isopropyl isostearate, cetyl 2-ethylhexanoate, cetostearyl 2-ethylhexanoate, stearyl 2-ethylhexanoate, hexyl isostearate, ethylene glycol dioctanoate, ethylene glycol dioleate, propylene glycol dicaprylate, propylene glycol dicaprylate/dicaprate, propylene glycol dicaprate, propylene glycol dioleate, neopentyl glycol dicaprate, neopentyl glycol dioctanoate, glyceryl tricaprylate, glyceryl tri 2-ethyl hexanoate, glyceryl tricaprylate/tricaprate, glyceryl tricaprylate/tricaprate/tristearate, glyceryl triundecylate, glyceryl triisopalmitate, glyceryl triisostearate, trimethylolpropane tri 2-ethylhexanoate, trimethylolpropane triisostearate, pentaerythrityl tetra 2-ethylhexanoate, pentaerythrityl tetramyristate, pentaerythrityl tetraisostearate, diglyceryl tetraisostearate, octyldodecyl neopentanoate, isocetyl octanoate, isostearyl octanoate, 2-ethylhexyl isopelargonate, hexyldecyl dimethyloctanoate, octyldodecyl dimethyloctanoate, 2-ethylhexyl isopalmitate, isocetyl isostearate, isostearyl isostearate, octyldodecyl isostearate, lauryl lactate, myristyl lactate, cetyl lactate, octyldodecyl lactate, triethyl citrate, acetyltriethyl citrate, acetyltributyl citrate, trioctyl citrate, triisocetyl citrate, trioctyldodecyl citrate, diisostearyl malate, 2-ethylhexyl hydroxystearate, di 2-ethylhexyl succinate, diisopropyl adipate, diisobutyl adipate, dioctyl adipate, diheptylundecyl adipate, sebacate diethyl, diisopropyl sebacate, dioctyl sebacate, cholesteryl stearate, cholesteryl isostearate, cholesteryl hydroxystearate, cholesteryl oleate, dihydrocholesteryl oleate, phytosteryl isostearate, phytosteryl oleate, isocetyl 12-stearoyl hydroxystearate, stearyl 12-stearoyl hydroxystearate, isostearyl 12-stearoyl hydroxystearate, polyoxyethylene (3) polyoxypropylene (1) cetyl ether acetate, polyoxyethylene (3) polyoxypropylene (1) isocetyl ether acetate, isononyl isononanoate, octyl isononanoate, tridecyl isononanoate and isotridecyl isononanoate;

silicone oils such as methyl polysiloxane, methylphenyl polysiloxane, methyl hydrogen polysiloxane, methyl cyclopolysiloxane, octamethyl cyclotetrasiloxane, decamethyl cyclopentasiloxane, dodecamethyl cyclohexasiloxane, octamethyl trisiloxane, decamethyl tetrasiloxane, tetradecamethyl hexasiloxane, highly polymerized methyl polysiloxane, dimethylsiloxane-methyl(polyoxyethylene)siloxane-methyl(polyoxypropylene)siloxane copolymer, dimethylsiloxane-methyl(polyoxyethylene)siloxane copolymer, dimethylsiloxane-methyl(polyoxypropylene)siloxane copolymer, dimethylsiloxane-methylcetyl oxysiloxane copolymer, dimethylsiloxane-methyl stearoxysiloxane copolymer, polyether modified silicone, alcohol modified silicone, alkyl modified silicone and amino modified silicone;

polyhydric alcohols such as ethylene glycol, diethylene glycol, triethylene glycol, polyethylene glycol, propylene glycol, dipropylene glycol, polypropylene glycol, pentanediol, glycerin, diglycerin, polyglycerin, isoprene glycol, 1,3-butylene glycol, 3-methyl-1,3-butanediol, 1,3-butanediol, 1,2-pentanediol and 1,2-hexanediol;

saccharides such as mannitol, sorbitol, xylitol, maltitol, erythritol, pentaerythritol, glucose, sucrose, fructose, lactose, maltose, xylose and trehalose;

polymers such as sodium alginate, carrageen, agar, furcellaran, guar gum, carrageenan quince seed, Amorphophalus konjak (arum root) mannan, tamarind gum, tara gum, dextrin, starch, locust bean gum, gum arabic, gum gatti, karaya gum, gum tragacanth, arabinogalactan, pectin, quince, chitosan, starch, curdlan, xanthan gum, gellan gum, cyclodextrin, dextran, pullulan, microcrystalline cellulose, methyl cellulose, ethyl cellulose, hydroxyethyl cellulose, hydroxypropyl cellulose, hydroxypropylmethyl cellulose, carboxymethyl cellulose, carboxy starch, cationized cellulose, starch phosphate ester, cationized guar gum, carboxymethyl-hydroxypropylated guar gum, hydroxypropylated guar gum, albumin, casein, gelatin, sodium polyacrylate, polyacrylic amide, carboxyvinyl polymer, polyethylene imine, highly polymerized polyethylene glycol, polyvinyl alcohol, polyvinyl pyrrolidone, polyvinyl ether, polyacryl amide, acrylic acid copolymer, methacrylic acid copolymer, maleic acid copolymer, vinylpyridine copolymer, ethylene/acrylic acid copolymer, vinyl pyrrolidone based polymer, vinyl alcohol/vinyl pyrrolidone copolymer, nitrogen-substituted acrylamide based polymer, amino modified silicone, cationized polymer, dimethylacryl ammonium based polymer, acrylic acid based anion polymer, methacrylic acid based anion polymer, modified silicone, acrylate/methacrylate alkyl (C 10 to 30) copolymer and polyoxyethylene/polyoxypropylene copolymer;

alcohols such as ethanol, isopropyl alcohol, 1-butanol, 2-butanol and benzyl alcohol;

anionic surfactants such as coconut oil fatty acid potassium, coconut oil fatty acid sodium, coconut oil fatty acid triethanolamine, potassium laurate, sodium laurate, triethanolamine laurate, potassium myristate, sodium myristate, isopropanolamine myristate, potassium palmitate, sodium palmitate, isopropanolamine palmitate, potassium stearate, sodium stearate, triethanolamine stearate, potassium oleate, sodium oleate, castor oil fatty acid sodium, zinc undecylate, zinc laurate, zinc myristate, magnesium myristate, zinc palmitate, zinc stearate, calcium stearate, magnesium stearate, aluminum stearate, calcium myristate, magnesium myristate, aluminum dimyristate, aluminum isostearate, polyoxyethylene lauryl ether acetate, sodium polyoxyethylene lauryl ether acetate, polyoxyethylene tridecyl ether acetate, sodium polyoxyethylene tridecyl ether acetate, sodium stearoyl lactate, sodium isostearoyl lactate, sodium lauroyl sarcosine, coconut oil fatty acid sarcosine, sodium coconut oil fatty acid sarcosine, coconut oil fatty acid sarcosine triethanolamine, lauroyl sarcosine, potassium lauroyl sarcosine, lauroyl sarcosine triethanolamine, oleoyl sarcosine, sodium myristoyl sarcosine, sodium stearoyl glutamate, coconut oil fatty acid acyl glutamic acid, potassium coconut oil fatty acid acyl glutamate, sodium coconut oil fatty acid acyl glutamate, coconut oil fatty acid acyl glutamate triethanolamine, lauroylacyl glutamic acid, potassium lauroylacyl glutamate, sodium lauroylacyl glutamate, lauroylacyl glutamate triethanolamine, myristoylacyl glutamic acid, potassium myristoylacyl glutamate, sodium myristoylacyl glutamate, stearoylacyl glutamic acid, potassium stearoylacyl glutamate, disodium stearoylacyl glutamate, sodium hydrogenated beef tallow fatty acid acyl glutamate, sodium coconut oil fatty acid/hydrogenated beef tallow fatty acid acyl glutamate, sodium coconut oil fatty acid methylalanine, lauroyl methylalanine, sodium lauroyl methylalanine, lauroyl methylalanine triethanolamine, sodium myristoyl methylalanine, sodium lauroyl methyltaurine, potassium coconut oil fatty acid methyltaurine, sodium coconut oil fatty acid methyltaurine, magnesium coconut oil fatty acid methyltaurine, sodium myristoyl methyltaurine, sodium palmitoyl methyltaurine, sodium stearoyl methyltaurine, sodium oleoyl methyltaurine, sodium alkane sulfonate, sodium tetradecene sulfonate, sodium sulfosuccinate dioctyl, disodium lauryl sulfosuccinate, sodium coconut oil fatty acid ethyl ester sulfonate, sodium lauryl sulfate, triethanolamine lauryl sulfate, sodium cetyl sulfate, triethanolamine alkyl (11,13,15) sulfate, sodium alkyl (12,13) sulfate, triethanolamine alkyl (12,13) sulfate, alkyl (12,14,16) ammonium sulfate, diethanolamine alkyl (12 to 13) sulfate, triethanolamine alkyl (12 to 14) sulfate, triethanolamine alkyl (12 to 15) sulfate, magnesium coconut oil alkyl sulfate/triethanolamine, lauryl ammonium sulfate, potassium lauryl sulfate, magnesium lauryl sulfate, monoethanolamine lauryl sulfate, diethanolamine lauryl sulfate, sodium myristyl sulfate, sodium stearylsulfate, sodium oleyl sulfate, triethanolamine oleyl sulfate, sodium polyoxyethylene lauryl ether sulfate, triethanolamine polyoxyethylene lauryl ether sulfate, sodium polyoxyethylene (1) alkyl (11,13,15) ether sulfate, triethanolamine polyoxyethylene (1) alkyl (11,13,15) ether sulfate, sodium polyoxyethylene (3) alkyl (11 to 15) ether sulfate, sodium polyoxyethylene (2) alkyl (12,13) ether sulfate, sodium polyoxyethylene (3) alkyl (12 to 14) ether sulfate, sodium polyoxyethylene (3) alkyl (12 to 15) ether sulfate, sodium polyoxyethylene (2) lauryl ether sulfate, sodium polyoxyethylene (3) myristyl ether sulfate, sodium higher fatty acid alkanol amide sulfate ester, lauryl phosphate, sodium lauryl phosphate, potassium cetyl phosphate, diethanolamine cetyl phosphate, polyoxyethylene oleyl ether phosphate, polyoxyethylene lauryl ether phosphate, sodium polyoxyethylene lauryl ether phosphate, polyoxyethylene cetyl ether phosphate, sodium polyoxyethylene cetyl ether phosphate, polyoxyethylene stearyl ether phosphate, polyoxyethylene oleyl ether phosphate, sodium polyoxyethylene oleyl ether phosphate, polyoxyethylene alkylphenyl ether phosphate, sodium polyoxyethylene alkylphenyl ether phosphate, triethanolamine polyoxyethylene alkylphenyl ether phosphate, polyoxyethylene octyl ether phosphate, polyoxyethylene (10) alkyl (12,13) ether phosphate, polyoxyethylene alkyl (12 to 15) ether phosphate, polyoxyethylene alkyl (12 to 16) ether phosphate, triethanolamine polyoxyethylene lauryl ether phosphate and diethanolamine polyoxyethylene oleyl ether phosphate;

cationic surfactants such as dioctylamine, dimethylstearylamine, trilaurylamine, diethylaminoethylamide stearate, lauryl trimethylammonium chloride, cetyl trimethylammonium chloride, cetyl trimethylammonium bromide, cetyl trimethylammonium saccharin, stearyl trimethylammonium chloride, alkyl (20 to 22) trimethylammonium chloride, lauryl trimethylammonium bromide, alkyl (16,18) trimethylammonium chloride, stearyl trimethylammonium bromide, stearyl trimethylammonium saccharin, alkyl (28) trimethylammonium chloride, di(polyoxyethylene) oleyl methylammonium (2EO) chloride, dipolyoxyethylene stearyl methylammonium chloride, polyoxyethylene (1) polyoxypropylene (25) diethylmethylammonium chloride, tri(polyoxyethylene) stearyl ammonium (5EO) chloride, distearyl dimethylammonium chloride, dialkyl (12 to 15) dimethylammonium chloride, dialkyl (12 to 18) dimethylammonium chloride, dialkyl (14 to 18) dimethylammonium chloride, dicocoyl dimethylammonium chloride, dicetyl dimethylammonium chloride, isostearyl lauryl dimethylammonium chloride, benzalkonium chloride, myristyl dimethylbenzyl ammonium chloride, lauryl dimethyl(ethylbenzyl) ammonium chloride, stearyl dimethylbenzyl ammonium chloride, lauryl pyridinium chloride, cetyl pyridinium chloride, lauroyl cholamino formylmethyl pyridinium chloride, stearoyl cholamino formylmethyl pyridinium chloride, alkyl isoquinolinium bromide, methyl benzethonium chloride and benzethonium chloride;

ampholytic surfactants such as 2-alkyl-N-carboxymethyl-N-hydroxyethyl imidazolium betaine, alkyldiamino ethyl glycine hydrochloride, sodium lauryldiamino ethyl glycine, sodium undecyl hydroxyethyl imidazolium betaine, undecyl-N-carboxymethyl imidazolium betaine, disodium coconut oil fatty acid acyl-N-carboxyethyl-N-hydroxyethyl ethylenediamine, disodium coconut oil fatty acid acyl-N-carboxyethoxyethyl-N-carboxyethyl ethylenediamine, disodium coconut oil fatty acid acyl-N-carboxymethoxyethyl-N-carboxymethyl ethylenediamine, sodium laurylamino propionate, sodium laurylamino dipropionate, triethanolamine laurylamino propionate, sodium palm oil fatty acid acyl-N-carboxyethyl-N-hydroxyethyl ethylenediamine, betaine lauryldimethylamino acetate, betaine coconut oil alkyldimethylamino acetate, betaine stearyl dimethylamino acetate, sodium stearyldimethyl betaine, coconut oil fatty acid amidopropyl betaine, palm oil fatty acid amidopropyl betaine, amidopropyl acetate betaine laurate, amidopropyl betaine ricinoleate, stearyl dihydroxyethyl betaine and lauryl hydroxysulfobetaine;

nonionic surfactants such as polyoxyethylene (10) alkyl (12,13) ether, polyoxyethylene lauryl ether, polyoxyethylene cetyl ether, polyoxyethylene stearyl ether, polyoxyethylene oleyl ether, polyoxyethylene (3,7,12) alkyl (12 to 14) ether, polyoxyethylene tridecyl ether, polyoxyethylene myristyl ether, polyoxyethylene-sec-alkyl (14) ether, polyoxyethylene isocetyl ether, polyoxyethylene cetostearyl ether, polyoxyethylene (2,10,20) isostearyl ether, polyoxyethylene oleylcetyl ether, polyoxyethylene (20) arachyl ether, polyoxyethylene octyldodecyl ether, polyoxyethylene behenyl ether, polyoxyethylene octylphenyl ether, polyoxyethylene nonylphenyl ether, polyoxyethylene dinonylphenyl ether, polyoxyethylene (1) polyoxypropylene (1,2,4,8) cetyl ether, polyoxyethylene (5) polyoxypropylene (1,2,4,8) cetyl ether, polyoxyethylene (10) polyoxypropylene (1,2,4,8) cetyl ether, polyoxyethylene (20) polyoxypropylene (1,2,4,8) cetyl ether, polyoxyethylene polyoxypropylene lauryl ether, polyoxyethylene (3) polyoxypropylene (34) stearyl ether, polyoxyethylene (4) polyoxypropylene (30) stearyl ether, polyoxyethylene (34) polyoxypropylene (23) stearyl ether, polyoxyethylene polyoxypropylene cetyl ether, polyoxyethylene polyoxypropylene decyltetradecyl ether, polyethylene glycol monolaurate, ethylene glycol monostearate, polyethylene glycol monostearate, polyethylene glycol monooleate, ethylene glycol fatty acid ester, self-emulsifying ethylene glycol monostearate, diethylene glycol laurate, polyethylene glycolmyristate, polyethylene glycol palmitate, diethylene glycol stearate, self-emulsifying polyethylene glycol (2) monostearate, polyethylene glycol isostearate, ethylene glycol dioctanoate, diethylene glycol dilaurate, polyethylene glycol dilaurate, polyethylene glycol (150) dipalmitate, ethylene glycol distearate, diethylene glycol distearate, polyethylene glycol distearate, ethylene glycol dioleate, polyethylene glycol dioleate, polyethylene glycol diricinoleate, polyoxyethylene (20) sorbitan monolaurate, polyoxyethylene (20) sorbitan monopalmitate, polyoxyethylene (6) sorbitan monostearate, polyoxyethylene (20) sorbitan monostearate, polyoxyethylene (20) sorbitan tristearate, polyoxyethylene (6) sorbitan monooleate, polyoxyethylene (20) sorbitan monooleate, polyoxyethylene (20) sorbitan trioleate, polyoxyethylene (20) coconut oil fatty acid sorbitan, polyoxyethylene (10 to 80) sorbitan monolaurate, polyoxyethylene sorbitan tristearate, polyoxyethylene (20) sorbitan isostearate, polyoxyethylene (150) sorbitan tristearate, polyoxyethylene castor oil, polyoxyethylene hydrogenated castor oil, polyoxyethylene (10) hydrogenated castor oil, polyoxyethylene (20) hydrogenated castor oil, polyoxyethylene (40) hydrogenated castor oil, polyoxyethylene (50) hydrogenated castor oil, polyoxyethylene (60) hydrogenated castor oil, lipophilic glycerin monostearate, lipophilic glycerin monooleate, self-emulsifying glycerin monostearate, coconut oil fatty acid glyceryl, glycerin laurate, glyceryl myristate, glyceryl isostearate, glyceryl ricinoleate, glyceryl monohydroxystearate, glycerin oleate, glyceryl linoleate, glyceryl erucate, glyceryl behenate, wheat germ oil fatty acid glyceride, safflower oil fatty acid glyceryl, hydrogenated soybean fatty acid glyceryl, saturated fatty acid glyceride, cotton seed oil fatty acid glyceryl, monomyristate glyceryl monoisostearate, mono tallowate glyceride, monolanolin fatty acid glyceryl, glyceryl sesquioleate, glyceryl distearate, glyceryl diisostearate, glyceryl diarachidate, sorbitan monolaurate, sorbitan monopalmitate, sorbitan monostearate, sorbitan monoisostearate, sorbitan monooleate, sorbitan sesquistearate, sorbitan sesquioleate, sorbitan tristearate, sorbitan trioleate, coconut oil fatty acid sorbitan, sorbitan isostearate, sorbitan sesquiisostearate, sorbitan distearate, diglyceryl isopalmitate, poly (4 to 10) glyceryl monolaurate, poly (10) glyceryl monomyristate, poly (2 to 10) glyceryl monostearate, poly(2 to 10)glyceryl monoisostearate, poly(2 to 10)glyceryl monooleate, diglyceryl sesquioleate, poly(2 to 10)glyceryl diisostearate, poly(6 to 10)glyceryl distearate, diglyceryl triisostearate, poly(10)glyceryl tristearate, poly(10)glyceryl trioleate, poly(2)glyceryl tetraisostearate, decaglyceryl pentastearate, poly(6 to 10)glyceryl pentaoleate, poly(10)glyceryl heptastearate, decaglyceryl decastearate, poly(10)glyceryl decaoleate, concentrated poly (6) glyceryl ricinoleate, sucrose fatty acid ester, coconut oil fatty acid sucrose ester, alkyl glucoside, coconut oil alkyl dimethylamine oxide, lauryl dimethylamine oxide, dihydroxyethyl lauryl dimethylamine oxide, stearyl dimethylamine oxide, oleyl dimethylamine oxide and polyoxyethylene coconut oil alkyl dimethylamine oxide;

natural surfactants such as saponin, lecithin, soybean phospholipid, hydrogenated soybean phospholipid, soybean lysophospholipid, hydrogenated soybean lysophospholipid, yolk lecithin, hydrogenated yolk lysophosphatidylcholine, phosphatidylcholine, phosphatidylethanolamine, phosphatidylserine, sphingophospholipid, sphingomyelin, ganglioside, bile acid, cholic acid, deoxycholic acid, sodium cholate, sodium deoxycholate, spiculisporic acid, rhamnolipid, trehalose lipid, sophorolipid and mannosyl erythritol lipid;

ultraviolet ray absorbers such as: para-aminobenzoic acid derivatives such as para-aminobenzoic acid, ethyl para-aminobenzoate, glyceryl para-aminobenzoate, amyl para-dimethyl aminobenzoate and 2-ethylhexyl para-dimethyl aminobenzoate; cinnamic acid derivatives such as benzyl cinnamate, mono-2-ethyl hexanoate glyceryl dipara-methoxycinnamate, methyl 2,4-diisopropyl cinnamate, ethyl 2,4-diisopropyl cinnamate, potassium para-methoxycinnamate, sodium para-methoxycinnamate, isopropyl para-methoxycinnamate, 2-ethylhexyl para-methoxycinnamate, 2-ethoxyethyl para-methoxycinnamate and ethyl para-ethoxycinnamate; urocanic acid derivatives such as urocanic acid and ethyl urocanate; benzophenone derivatives such as 2,4-dihydroxybenzophenone, 2,2′,4,4′-tetrahydroxybenzophenone, sodium 2-hydroxy-4-methoxy-5-sulfobenzophenone, 2-hydroxy-4-methoxybenzophenone-5-sulfonate, 2-hydroxy-4-methoxybenzophenone, 2,2′-dihydroxy-4,4′-dimethoxybenzophenone and sodium 2,2′-dihydroxy-4,4′-dimethoxy-5-sulfobenzophenone; salicylic acid derivatives such as ethylene glycol salicylate, salicylate-2-ethylhexyl, phenyl salicylate, benzyl salicylate, p-tert-butylphenyl salicylate, homomethyl salicylate and salicylate-3,3,5-trimethylcyclohexyl; 2-(2′-hydroxy-5′-methoxyphenyl)benzotriazole and 4-tert-butyl-4′-methoxybenzoyl methane;

powders and color materials such as: kaolin, silicic anhydride, magnesium aluminum silicate, sericite, talc, boron nitride, mica, montmorillonite, hemp cellulose powder, wheat starch, silk powder, maize starch; natural dyes such as nitro dyes, azo dyes, nitroso dyes, triphenylmethane dyes, xanthene dyes, quinoline dyes, anthraquinone dyes, indigo dyes, pyrene dyes, phthalocyanine dyes, flavonoid, quinone, porphyrin, water soluble annatto, sepia powder, caramel, guaiazulene, gardenia blue, gardenia yellow, cochineal, shikonin, sodium copper chlorophyllin, paprika dye, safflower red, safflower yellow, laccaic acid and riboflavin butyrate ester; carbon black, yellow iron oxide, black iron oxide, red iron oxide, iron blue, ultramarine blue, zinc oxide, chromiumoxide, titanium oxide, black titanium oxide, zirconium oxide, chromium hydroxide, alumina, magnesium oxide, barium sulfate, aluminum hydroxide, calcium carbonate, lithium cobalt titanate, manganese violet and pearl pigment.

plant extracts such as Angelica keiskei extract, Uncaria gambir extract, avocado extract, sweet hydrangea leaf extract, Gynostemma pentaphyllum makino extract, Althaea officinalis extract, Arnica montana extract, oil soluble Arnica montana extract, almond extract, aloe extract, Japanese styrax benzoin extract, Ginkgo biloba extract, Stinging nettle extract, Orris rhizome root extract, fennel extract, turmeric extract, dog rose fruit extract, Echinacea leaf extract, Scutellaria root extract, Phellodendron bark extract, Japanese captis extract, barley extract, okura extract, Hypericum perforatum extract, oil soluble Hypericum perforatum extract, Lamium album extract, oil soluble Lamium album extract, Ononis spinosa root extract, Nasturtium officinale extract, orange extract, orange flower water, seaweed extract, persimmon tannin, pueraria root extract, Japanese valerian extract, cattail extract, Chamomile (matricaria) extract, oil soluble Chamomile (matricaria) extract, Chamomile (matricaria) distillate, Avena sativa (oat) kernel extract, carrot extract, oil soluble carrot extract, carrot oil, Artemicia capillaris extract, Glycyrrhiza glabra (licorice) extract, powdered Glycyrrhiza glabra (licorice) extract, Glycyrrhiza glabra (licorice) extract flavonoid, cantharides tincture, raspberry extract, kiwi extract, cinchona extract, cucumber extract, apricot kernel extract, quince seed extract, gardenia florida extract, Sasa albomarginata extract, Sophora root extract, walnut shell extract, Citrus paradisi (grapefruit) extract, Clematis vitalba leaf extract, black sugar extract, chlorella extract, mulberry bark extract, Cinnamon bark extract, Gentian extract, Geranium herb extract, black tea extract, Nuphar extract, burdock root extract, oil soluble burdock root extract, wheat germ extract, hydrolyzed wheat powder, rice bran extract, fermented rice bran extract, Symphytum officinale (comfrey) extract, Asiasarum root extract, Crocus sativus (saffron) extract, Saponaria officinalis extract, oil soluble salvia extract, Crataegus cuneata fruit extract, Zanthoxylum fruit extract, Lentinus edodes extract, powdered Lentinus edodes extract, Rehmannia root extract, Lithospermum root extract, oil soluble Lithospermum root extract, Perilla herb extract, linden extract, oil soluble Tilia europaea extract, Filipendula extract, Peony root extract, Coix lacryma-jobi extract, ginger extract, oil soluble ginger extract, ginger tincture, Acorus calamus root extract, Betula pendula (birch) extract, oil soluble Betula alba (birch) extract, Betula pendula (birch) sap, Lonicera japonica extract, Equisetum arvense extract, oil soluble Equisetum arvense extract, scordinin, stevia extract, ivy extract, Crataegus oxyacantaha (white thorn) extract, sambucus extract, Juniperus communis extract, Achillea milefolium extract, oil soluble Achillea milefolium extract, Mentha piperita (peppermint) extract, Salvia officinalis (sage) extract, oil soluble Salvia officinalis (sage) extract, Salvia officinalis (sage) water, Malva Sylvestris (mallow) extract, Apium graveolens (celery) extract, Cnidium officinale extract, Cnidium officinale water, Swertia herb extract, Glycine max (soybean) extract, Jujube extract, thyme extract, green tea extract, tea leaf dry distilled solution, tea seed extract, clove extract, Citrus unshiu peel extract, Camellia japonica extract, Centella asiatica extract, oil soluble walnut extract, duku extract, Terminalia sericea extract, Capsicum tincture, Japanese angelica root extract, oil soluble Japanese angelica root extract, Japanese angelica root water, Calendula officinalis flower extract, oil soluble Calendula officinalis flower extract, soy milk powder, peach seed extract, Bitter orange peel extract, Houttuynia cordata extract, Solanum lycopersicum (tomato) extract, Potentilla tormentilla Schrk (Rosaceae) extract, fermented soybeans extract, Ginseng extract, oil soluble Ginseng extract, Allium sativum (garlic) extract, wild rose extract, oil soluble wild rose extract, malt extract, malt root extract, Ophiopogon tuber extract, parsley extract, rye leaf juice concentrate, peppermint distillate, witch hazel distillate, witch hazel extract, rose extract, parietaria extract, Isodonis japonicus extract, Eriobotrya japonica leaf extract, oil soluble Eriobotrya japonica leaf extract, coltsfoot extract, hoelen extract, Ruscus aculeatus root extract, powdered Ruscus aculeatus root extract, grape extract, grape leaf extract, grape water, Hayflower extract, Luffa cylindrica fruit extract, Luffa cylindrica fruit water, Carthamus tinctorius (safflower) extract, oil soluble Tilia platyphyllos extract, linden distillate, Paeonia suffruticosa (peony) extract, Humulus lupulus (hops) extract, oil soluble Humulus lupulus (hops) extract, pine extract, Silybum marianum (milk thistle) extract, Aesculus hippocastanum (horse chestnut) extract, oil soluble Aesculus hippocastanum (horse chestnut) extract, Sapindus mukurossi extract, Melissa officinalis (balm mint) extract, Melilotus officinalis (melilot) extract, Prunus persica (peach) leaf extract, oil soluble Prunus persica (peach) leaf extract, bean sprouts extract, Centaurea cyanus flower extract, Centaurea cyanus flower distillate, Eucalyptus globulus extract, Saxifrage extract, Lilium (lily) extract, Coix seed extract, oil soluble Coix seed extract, Artemisia princeps pampanini extract, Artemisia princeps pampanini water, Lavandula angustifolia (lavender) extract, Lavandula angustifolia (lavender) water, apple extract, Ganoderma lucidum extract, Lactuca sativa (lettuce) extract, lemon extract, Astragalus sinicus extract, Rosa centifolia (rose) flower water, Rosemarinus officinalis (rosemary) extract, oil soluble Rosemarinus officinalis (rosemary) extract, Anthemis nobilis extract and Sanguisorba officinalis extract;

amino acids and peptides such as glycine, alanine, valine, leucine, isoleucine, serine, threonine, phenylalanine, tyrosine, tryptophan, cystine, cysteine, methionine, proline, hydroxyproline, aspartic acid, asparagine, glutamic acid, glutamine, arginine, histidine, lysine, γ-aminobutyric acid, DL-pyrrolidonecarboxylic acid, ε-aminocaproic acid, hydrolyzed elastin, water soluble elastin, hydrolyzed collagen, water soluble collagen, casein, glutathione, wheat peptides and soybean peptide;

vitamins and factors acting like a vitamin such as: vitamin A and analogues thereof such as retinol, retinal, retinoic acid, retinol acetate and retinol palmitate; carotenoids such as α-carotene, β-carotene, γ-carotene, 6-carotene, lycopene, zeaxanthin, cryptoxanthin, echinenon and astaxanthin; vitamin B₁ and analogues thereof such as thiamines; vitamin B₂ and analogues thereof such as riboflavin; vitamin B₆ and analogues thereof such as pyridoxine, pyridoxal and pyridoxamine; vitamin B₁₂ and analogues thereof such as cyanocobalamin; folic acids, nicotinic acid, nicotinamide, pantothenic acids, biotins; vitamin C and analogues thereof such as L-ascorbic acid, sodium L-ascorbate, L-ascorbyl stearate, L-ascorbyl palmitate, L-ascorbyl dipalmitate, L-ascorbyl tetraisopalmitate, disodium L-ascorbate sulfate ester, magnesium L-ascorbyl, sodium L-ascorbyl phosphate, L-ascorbate-2-glucoside and magnesium L-ascorbate-2-phosphate; vitamin D and analogues thereof such as ergocalciferol and cholecalciferol; vitamin E and analogues thereof such as d-α-tocopherol, DL-α-tocopherol, dl-α-tocopherol acetate, dl-α-tocopherol succinate, β-tocopherol, γ-tocopherol and d-δ-tocopherol; ubiquinones, vitamin K and analogues thereof, carnitine, ferulic acid, γ-oryzanol, α-lipoic acid and orotic acid;

antiseptic agents such as benzoic acid, sodium benzoate, undecylenic acid, salicylic acid, sorbic acid, potassium sorbate, dehydroacetic acid, sodium dehydroacetate, isobutyl paraoxybenzoate, isopropyl paraoxybenzoate, ethyl paraoxybenzoate, butyl paraoxybenzoate, propyl paraoxybenzoate, benzyl paraoxybenzoate, methyl paraoxybenzoate, sodium paraoxybenzoate methyl, phenoxyethanol, light sensitive dye No. 101, light sensitive dye No. 201 and light sensitive dye No. 401;

antioxidizing agents such as butylhydroxyanisole, butylhydroxytoluene, propyl gallate, erythorbic acid, sodium erythorbate, para-hydroxyanisole and octyl gallate;

chelating agents to bind to a metal ion such as trisodium ethylenediamine hydroxyethyl triacetate, edetic acid, disodium edetate, trisodium edetate, tetrasodium edetate, sodium citrate, gluconic acid, phytic acid, sodium polyphosphate and sodium metaphosphate;

moisturizing agents such as hyaluronic acid, sodium hyaluronate, sodium chondroitin sulfate, sodium lactate, sodium pyrrolidone carboxylate, betaine, lactic acid bacteria fermented solution, yeast extract and ceramide;

anti-inflammatory agents such as glycyrrhizic acid, trisodium glycyrrhizinate, dipotassium glycyrrhizinate, monoammonium glycyrrhizinate, β-glycyrrhetinic acid, glycerin glycyrrhetinate, stearyl glycyrrhetinate, lysozyme chloride, hydrocortisone and allantoin;

pH adjusting agents such as sodium hydroxide, potassium hydroxide and triethanolamine;

salts such as sodium chloride, potassium chloride, magnesium chloride and sodium sulfate;

α-hydroxy acids such as citric acid, glycolic acid, tartaric acid and lactic acid;

whitening agents such as arbutin, α-arbutin and placenta extract;

essential oils such as Archangelica officinalis (angelica) oil, Canangium odoratum (ylangylang) oil, Canarium luzonicum (elemi) oil, orange oil, Chamomilla recutita (matricaria) oil, Anthemis nobilis oil, Elettaria cardamom (cardamon) oil, Acorus calamus (calamus) oil, Ferula galbaniflua (galbanum) oil, Cinnamomum camphora (camphor) oil, Daucus carota (carrot) seed oil, Salvia sclarea (clary sage) oil, Citrus paradisi (grapefruit) oil, Eugenia caryophyllus (clove) oil, Cinnamon bark oil, Coriandrum sativum (coriander) oil, Cupressus sempervirens (cypress) oil, Santalum album (sandalwood) oil, Juniperus virginiana (cedarwood) oil, Cympogon nardus (citronella) oil, Cinnamomum zeylanicum (Cinnamon) leaf oil, Jasmine officinale (jasmine) absolute oil, Juniperus communis (juniper Berry) oil, Zingiber officinale (ginger) extract, Mentha spicata (spearmint) oil, Salvia officinalis (sage) oil, cedar oil, Pelargonium grabeolens (geranium) oil, Thymus vulgaris (thyme) oil, Melaleuca alternifolia (tea tree) oil, Myristica fragrans (nutmeg) oil, Melaleuca qui.viridiflara (niaouli) oil, Citrus aurantium (neroli) oil, pine oil, Ocimum basilicum (basil) oil, Mentha arvensis oil, Pogostemon patchouli (patchouli) oil, Cymbopogon martini (palmarosa) oil, Foeniculum vulgare (fennel) oil, Citrus bigaradia (petitgrain) oil, Piper nigrum (black pepper) oil, Boswellia carterii (frankincense) oil, Vetiveria zizanoides (vetivert) oil, Mentha piperita (peppermint) oil, Citrus bergamia (bergamot) oil, benzoin oil, Aniba rosaeodora (bois de rose) oil, Origanum majorana (marjoram) oil, mandarin oil, Conumiphora myrrha (myrrh) oil, Melissa officinalis (balm mint) oil, Eucalyptus globulus oil, Citrus junos oil, Citrus aurantifolia (lime) oil, Ravensare aromaticum (ravensare) oil, Lavandula latifolia (lavandin) oil, Lavandula angustifolia (lavender) oil, Tilia vulgaris (linden) oil, lemon oil, lemon grass oil, rose oil, Aniba rosaeodora (rosewood) oil, Rosemarinus officinalis (rosemary) oil and Levisticum officinale (lovage) oil;

terpenes such as limonene, pinene, terpinene, terpinolene, myrcene and longifeelene;

fragrance, water, and the like.

Furthermore, to the external preparation for skin of the invention may also be added any existing raw material of cosmetics at a general concentration. All raw materials of cosmetics described in, for example, Keshouhin genryou kizyun (Standards of raw materials of cosmetics), second edition, notes, edited by Society of Japanese Pharmacopoeia, 1984 (YAKUJI NIPPO LIMITED.), Keshouhin genryou kizyun-gai seibun kikaku (Standards of raw materials of cosmetics, nonstandard ingredients), under the editorship of Pharmaceutical Affairs Bureau Evaluation and Registration Division, 1993 (YAKUJI NIPPO LIMITED.), Keshouhin genryou kizyun-gai seibun kikaku tsuiho (Standards of raw materials of cosmetics, nonstandard ingredient Supplement), under the editorship of Pharmaceutical Affairs Bureau Evaluation and Registration Division, 1993 (YAKUJI NIPPO LIMITED.), Keshouhin syubetsu kyoka kizyun (Standards of cosmetic classification permission), under the editorship of Pharmaceutical Affairs Bureau Evaluation and Registration Division, 1993 (YAKUJI NIPPO LIMITED.), Keshouhin syubetsu haigou seibun kikaku (Standards of cosmetic classification ingredients), under the editorship of Pharmaceutical Affairs Bureau Evaluation and Registration Division, 1997 (YAKUJI NIPPO LIMITED.), Keshouhin genryou jiten (Dictionary of raw materials of cosmetics), 1991 (Nikko Chemicals Co., Ltd.), Atarashii Keshouhinkinou Sozai 300 (New 300 raw materials having cosmetic functions), 2002 (CMC Publishing Co., Ltd) and the like may be used.

### BEST MODE FOR CARRYING OUT THE INVENTION

Hereunder, the present invention will be specifically explained with reference to the Examples, however, the present invention is not limited to these Examples.

### Example 1

Under a nitrogen atmosphere, 1,6-anhydro-β-D-glucopyranose (manufactured by Tokyo Kasei Co., Ltd., 13.0 g), dried propylene carbonate (manufactured by Aldrich Co., 12.5 ml) and 66% by mass of 2-butynyl-tetramethylene sulfonium hexafluoroantimonate (manufactured by Asahi Denka Co., Ltd., 65.8 μl) were charged into a schlenk tube which had been thoroughly dried. Then, this schlenk tube was placed in an oil bath, and the oil bath was heated to 100° C., whereby 1,6-anhydro-β-D-glucopyranose was well dissolved. Then, the temperature was raised up to 130° C., whereby polymerization was started while well stirring the mixture. After a 30-minute reaction time, the polymerization solution was poured into methanol to stop the polymerization. After the solvent was distilled off, reprecipitation was repeated with water and methanol. Further, dialysis was performed for purification, and by freeze-drying, a white powder of multi-branched polysaccharide A (5.3 g) was obtained. The yield was 41%. The structure of this multi-branched polysaccharide A was confirmed by ¹H-NHR and ¹³C-NMR. In addition, the weight average molecular weight was 20,000 (the light scattering method) and the branching degree was 0.38.

### Example 2

The multi-branched polysaccharide A (1.0 g) obtained in the Example 1, 1, 8-diisocyanate octane (0.2 g) and dried pyridine (10 ml) were put into a flask which had been thoroughly dried, and reacted at 100° C. for 3 hours while well stirring. After the reaction, the reaction solution was poured into methanol, the solvent was distilled off, and reprecipitation was repeated with water and methanol. Then, by freeze-drying, a white powder of polymer compound B (1.1 g) was obtained. The 1% aqueous solution of this polymer compound B was transparent.

### Example 3

Under a nitrogen atmosphere, isocyanate ethyl methacrylate (manufactured by Showa Denko K.K., 204 mg), methyl methacrylate (manufactured by Aldrich Co., 903 mg) and azobisisobutyronitrile (AIBN, 50.8 mg) were put into a pressure-resistant flask which had been thoroughly dried, dimethylformamide (DMF, 10 ml) was added, and thoroughly dissolved by well stirring. Then, this flask was subjected to an operation of degassing under vacuum and introduction of dried nitrogen 5 times. Finally the flask was degassed under vacuum and sealed. Then, this flask was placed in an oil bath at 60° C. and reaction was performed for 20 hours. After the reaction, the flask was opened after the temperature was lowered sufficiently, then the reaction solution was poured into hexane. After the hexane was removed, a small amount of chloroform was added and the residue was dissolved, which was added to hexane again for reprecipitation. This reprecipitation operation was repeated, and the precipitation was recovered by filtration. Then by freeze-drying, a white powder of polymer C (1.07 g) was obtained. The yield was 97%. The structure of this polymer C was confirmed by FT-IR, ¹H-NHR and ¹³C-NMR. In addition, the weight average molecular weight was 13,900 (GPC/THF, in terms of polystyrene).

### Example 4

The multi-branched polysaccharide A (1.0 g) obtained in Example 1, the polymer C (1.0 g) obtained in Example 3, and dried pyridine (10 ml) were put into a flask which had been thoroughly dried, and reacted at 100° C. for 3 hours while well stirring. After the reaction, the reaction solution was poured into methanol, the solvent was distilled off, and reprecipitation was repeated with water and methanol. Then, by freeze-drying, a white powder of polymer compound D (1.1 g) was obtained. The 1% aqueous solution of this polymer compound was transparent.

### Example 5

Emulsions having compositions as shown in Table 1 were prepared by conventional methods. Ten females of age 20-30 used the emulsions, and their feedbacks after using each emulsion is shown in Table 2. It was demonstrated that by using sample 1-1 containing the polymer compound D obtained in Example 4, a moist skin feeling could be maintained. In addition, a rough feeling sample 1-2 gave showed it slow solubility.

TABLE 1

| | Sample | | |
|---|---|---|---|
| | 1-1 | 1-2 | 1-3 |
| Polymer compound D | 1.0% | — | — |
| Polymethyl methacrylate | — | 1.0% | — |
| Glycerin | 8.0% | 8.0% | 8.0% |
| 1,3-butylene glycol | 2.0% | 2.0% | 2.0% |
| Sodium citrate | 1.0% | 1.0% | 1.0% |
| Polyethylene glycol-50 | 0.5% | 0.5% | 0.5% |
| Methylparaben | 0.2% | 0.2% | 0.2% |
| Purified water | 87.3% | 87.3% | 88.3% |
| | 100.0% | 100.0% | 100.0% |

TABLE 2

| | Sample | | |
|---|---|---|---|
| | 1-1 | 1-2 | 1-3 |
| Moisture feeling after use | 9 | 3 | 2 |
| Rough feeling while using | 2 | 7 | 0 |
| No change of feeling | 1 | 7 | 8 |

### Example 6

The emulsions having compositions as shown in Table 1 were applied on the flexor surface of the forearm of three females aged of age 30 to 40 twice a day, once in the morning and the other time in the evening for 7 days. The moisture levels in the corneum layer were measured and compared on the morning of the eighth day with a conductivity value determined through high-frequency inductance method by using an impedance meter (SKICON-200: manufactured by IBS Co., Ltd.) as an index, and the results were shown in Table 3. According to the results, by using sample 1-1 containing polymer compound D obtained in Example 4, enhancement in conductivity was observed, which indicated that the moisture in the corneum layer was increased to replenish the skin moisture.

TABLE 3

| | Before | After 1-1 | After 1-2 | After 1-3 |
|---|---|---|---|---|
| | applying | applied | applied | applied |
| Conductivity | 100 | 187 | 155 | 122 |

### INDUSTRIAL APPLICABILITY

The polymer compound of the present invention comprises a repeating structural unit which imparts a moisture retention property to an external preparation for skin and further comprises in the same molecule a structure imparting viscosity or solubility. By blending the polymer compound, an excellent external preparation for skin which imparts moisture to the skin, gives a good feeling to the skin and which is less likely to generate separation, precipitation and crystallization, can be provided.

1. A moisture retention polymer compound comprising a structural unit including a multi-branched polysaccharide derivative in which the hydroxyl group (OH) may be substituted by OR wherein R represents a hydrogen atom, a hydrocarbon having 1 to 30 carbon atoms, or a hydrocarbon having 1 to 30 carbon atoms and a hetero atom, and a structural unit without a multi-branched polysaccharide derivative.

2. The moisture retention polymer compound according to claim 1, wherein the main chain of the polymer compound consists of a structural unit including a multi-branched polysaccharide derivative.

3. The moisture retention polymer compound according to claim 2, which is a polymer compound obtained by copolymerization of a multi-branched polysaccharide derivative having at least two hydroxyl groups and a dicarboxylic acid and/or a diisocyanate.

4. The moisture retention polymer compound according to claim 1, having a structural unit including a multi-branched polysaccharide derivative in the side chain of the polymer compound.

5. The moisture retention polymer compound according to claim 4, which is a polymer compound obtained by reacting a polymer compound having a carboxyl group and/or an isocyanate group in the main chain with the hydroxyl group (OH) of the multi-branched polysaccharide derivative.

6. The moisture retention polymer compound according to claim 1, wherein the multi-branched polysaccharide derivative is a polymer including as a monomer an anhydrosaccharide and/or an anhydrosaccharide derivative with at least one of the hydroxyl groups (OH) of the anhydrosaccharide being substituted by OR wherein R represents a hydrogen atom, a hydrocarbon having 1 to 30 carbon atoms, or a hydrocarbon having 1 to 30 carbon atoms and a hetero atom.

7. The moisture retention polymer compound according to claim 6, wherein the anhydrosaccharide or the anhydrosaccharide portion of the anhydrosaccharide derivative is at least one kind selected from the group consisting of 1,6-anhydrosaccharide, 1,4-anhydrosaccharide, 1,3-anhydrosaccharide, 1,2-anhydrosaccharide, and 5,6-anhydrosaccharide.

8. The moisture retention polymer compound according to claim 1, in which the branching degree of the multi-branched polysaccharide derivative is 0.05 to 1.00.

9. The moisture retention polymer compound according to claim 1, wherein solubility of the polymer compound in water, ethanol or a mixed solution of water and ethanol is 1.0% by mass or higher.

10. A moisturizer comprising a polymer compound according to claim 1.

11. An external preparation for skin comprising 0.1 to 99.9% by mass of a polymer compound according to claim 1.

12. A cosmetic product containing the moisturizer according to claim 10.

13. A cosmetic product containing an external preparation for skin according to claim 11.
